(19) United States
(12) Patent Application Publication
(10) Pub. No.: US 2021/0137793 A1
(43) Pub. Date: May 13, 2021

(54) ACTIVE INSERTED GASTRIC TUBE WITH AN INTRA-BODY COMMUNICATION FUNCTION

(71) Applicants: NANTONG UNIVERSITY, Nantong (CN); QIDONG HOSPITAL OF TCM, Nantong (CN)

(72) Inventors: Guiling GENG, Nantong (CN); Yanling LU, Nantong (CN); Xuehua HAN, Nantong (CN); Zihan GENG, Nantong (CN); Jian WANG, Nantong (CN)

(21) Appl. No.: 17/148,744

(22) Filed: Jan. 14, 2021

Related U.S. Application Data
(63) Continuation of application No. PCT/CN2018/117764, filed on Nov. 27, 2018.

(30) Foreign Application Priority Data
Oct. 18, 2018 (CN) 201811215629.2

(51) Int. Cl. A61J 15/00

(52) U.S. Cl. CPC A61J 15/0084; A61J 2200/70; A61J 15/0003

(57) ABSTRACT

Disclosed is an active inserted gastric tube with an intra-body communication function, including a tube body, a pulse module, an intra-body communication module and a control module. The pulse module is arranged at a front end of the tube body, and is configured to generate a pulse signal. the intra-body communication module is configured to receive the pulse signal generated by the pulse module and transmit the pulse signal to the control module. The control module is configured to analyze the received pulse signal. In this way, the pulse signal is generated by the pulse module, and the gastric tube is inserted into the esophagus through the tube body. The pulse signal is transmitted through a human body to realize an intra-body communication. The control module analyzes the pulse signal transmitted through the human body, so as to identify whether the tube body is inserted into the esophagus or trachea.

### CROSS-REFERENCE TO RELATED APPLICATIONS

This application is a continuation of International Patent Application No. PCT/CN2018/117764, filed on Nov. 27, 2018, which claims the benefit of priority from Chinese Patent Application No. 201811215629.2, filed on Oct. 18, 2018. The content of the aforementioned applications, including any intervening amendments thereto, is incorporated herein by reference in its entirety.

### TECHNICAL FIELD

The present disclosure relates to medical supplies and equipment, and more particular to an active inserted gastric tube with an intra-body communication function.

### BACKGROUND

Gastric tube insertion is a necessary clinical skill for medical and nursing students. The trachea and esophagus are adjacent. Generally, during the gastric tube insertion for the critically ill patients or the patients with dysphagia, the cartilago epiglottica will cover the trachea. However, when the patient is in a critical condition and the epiglottic cartilage cannot cover the trachea in time, the gastric tube may be mistakenly inserted into the trachea due to the improper operation. In addition, some elderly patients with dysphagia need an indwelling gastric tube. When the body position changes, the indwelling gastric tube may be released and enter the trachea, and the patient may have some symptoms such as cough, anhelation, feeling suddenly oppressed and aspiration pneumonia, or even worse, the patient may suffocate or die.

### SUMMARY

An object of the present disclosure is to provide an active inserted gastric tube with an intra-body communication function, so as to solve the above-mentioned problems.

The present disclosure provides an active inserted gastric tube with an intra-body communication function, comprising:

a tube body;

a pulse module;

an intra-body communication module; and

a control module;

wherein the pulse module is arranged at a front end of the tube body, and is configured to generate a pulse signal;

the intra-body communication module is configured to receive the pulse signal generated by the pulse module and transmit the pulse signal to the control module; and

the control module is configured to analyze the received pulse signal.

The gastric tube generates the pulse signal through the pulse module, and is inserted into esophagus through the tube body. The pulse signal is transmitted through a human body to realize an intra-body communication. The control module analyzes the pulse signal transmitted through the human body, so as to identify whether the tube body is inserted into the esophagus or trachea.

In some embodiments, the pulse module comprises a pulse generator, a first electrode and a first pulse amplifier; the pulse generator is electrically connected to the first pulse amplifier; the first pulse amplifier is electrically connected to the first electrode; and the first electrode is arranged at the front end of the tube body.

In some embodiments, a channel is arranged in a wall of the tube body; a wire is arranged inside the channel; the first pulse amplifier is electrically connected to the first electrode through the wire; the first electrode is circular; and the first electrode is coated on an edge of the front end of the tube body.

In some embodiments, the intra-body communication module comprises a human body surface, an esophagus and a trachea; the tube body is insertable into the esophagus; and the control module is in communication connection with the human body surface.

In some embodiments, the control module comprises a microprocessor, a second pulse amplifier and a second electrode; the second electrode is sheet, and is attached to the human body surface; the second electrode is electrically connected to the second pulse amplifier; and the second pulse amplifier is electrically connected to the microprocessor.

### BRIEF DESCRIPTION OF THE DRAWINGS

FIG. 1 depicts a perspective view of an active inserted gastric tube with an intra-body communication function in accordance with the present disclosure; and

FIG. 2 is a response curve of a resistance-capacitance network activated by a step function in accordance with the present disclosure.

### DETAILED DESCRIPTION OF EMBODIMENTS

The present disclosure will be further described below in detail with reference to the accompanying drawings.

As shown in FIG. 1, an active inserted gastric tube with an intra-body communication function includes a tube body 1, a pulse module 2, an intra-body communication module 3 and a control module 4.

The pulse module 2 is arranged at a front end of the tube body 1, and is configured to generate a pulse signal.

The intra-body communication module 3 is configured to receive the pulse signal generated by the pulse module 2 and transmit the pulse signal to the control module 4.

The control module 4 is configured to analyze the received pulse signal.

The pulse module 2 includes a pulse generator 21, a first electrode 22 and a first pulse amplifier 23. The pulse generator 21 is electrically connected to the first pulse amplifier 23. The first pulse amplifier 23 is electrically connected to the first electrode 22, and the first electrode 22 is arranged at the front end of the tube body 1.

A channel 11 is arranged in a wall of the tube body 1, and a wire is arranged inside the channel 11. The first pulse amplifier 23 is electrically connected to the first electrode 22 through the wire. The first electrode 22 is circular, and the first electrode 22 is coated on an edge of the front end of the tube body 1.

The intra-body communication module 3 includes a human body surface 31, an esophagus 32 and a trachea 33. The tube body 1 is insertable into the esophagus 32. The control module 4 is in communication connection with the human body surface 31.

The control module 4 includes a microprocessor 41, a second pulse amplifier 42 and a second electrode 43. The second electrode 43 is sheet, and is attached to the human body surface 31. The second electrode 43 is electrically connected to the second pulse amplifier 42. The second pulse amplifier 42 is electrically connected to the microprocessor 41. The microprocessor 41 can also be electrically connected to the pulse generator 21 for controlling the pulse generator 21 to turn on and off.

It should be noted that the above-mentioned electrical connection and communication connection are all connected by wires.

In an embodiment, in order to realize an intra-body communication, the human body is regarded as a communication channel with a continuous medium, and the communication channel includes bone, muscle, fat and skin from the inside to the outside. The human body is an electromagnetic compatibility system with electrical conductivity and dielectric constant. The electrical conductivity and dielectric constant of the human body determine the magnitude of the current and the amplitude of polarization, respectively. The layers of bone, muscle, fat and skin have specific electrical conductivities and dielectric constants, respectively.

The dielectric constant is obtained according to the following formula:

$$ɛ ( ω ) = {ɛ}_{∞} + \frac{{ɛ}_{s} - {ɛ}_{∞}}{1 + {( j ωτ )}^{1 - a}}$$

in the formula, ω is an angular frequency of electromagnetic wave; τ is relaxation time; α is a weighting factor; εₛ is a dielectric constant when ωτ<<1; ε_{∞} is a dielectric constant when ωτ>>1; and ε(ω) is a dielectric constant being a function of frequency.

The electrical conductivity is obtained according to the following formula:

$$σ ( ω ) = \frac{Im [ ɛ ( ω ) ]}{ω}$$

in the formula, Im[ε(ω)] is an imaginary part of the dielectric constant corresponding to different frequencies; ω is an angular frequency of electromagnetic wave; and σ(ω) is an electrical conductivity being a function of frequency.

The trachea and the esophagus have different tissue structures.

The trachea includes rings of hyaline cartilage wrapped by an elastic fiber membrane. The esophagus includes an outer layer of fiber, a layer of muscle, a layer connective tissue and a layer mucosa.

Since the trachea and the esophagus have different tissue structures, their characteristic impedances are also different.

Electrical impedance (including resistance and reactance) quantitatively describes how much current is blocked, and it is a comprehensive expression of all the blocking forms of ion flow. When a biological tissue is introduced into an electric field, there may be two main reactions: a movement of charged ions along a direction of the electric field and a polarization of stationary particles. Therefore, the electrical impedance consists of two parts: a resistance caused by the movement of charged ion and a reactance caused by the polarization of the stationary particles. The resistance is usually caused by a friction generated by moving ions (such as sodium and chloride ions). The reactance is usually caused by the polarization of stationary molecules (such as cell membranes and protein molecules, which are similar to a dielectric material between two metal plates of a capacitor).

Therefore, an influence of a capacitance effect during the intra-body communication is not negligible.

In circuit theory, a voltage of a capacitor is calculated as follows:

$$v = \frac{1}{c} {\int }_{0}^{t} idt$$

in the formula, c represents a capacitance of the capacitor.

When one or more excitation sources act on a network, many responses are generated in the network. The network is often activated by a step function. A response curve of a resistance-capacitance (R-C) network activated by a step function is shown in FIG. 2.

The electrical impedance of the human body (including resistance and reactance) can be equivalent to a R-C network, and the resistance and capacitance of different tissues are different. The method used herein for identifying the trachea and the esophagus is based on the fact that the trachea and the esophagus have different tissue structures, and their resistance and capacitance are also different. When the step function acts on different R-C networks, the response curves are different. The microprocessor 41 analyzes the response curves to determine the type of tissue (trachea or esophagus).

In summary, the gastric tube generates a pulse signal through the pulse module, and is inserted into the esophagus through the tube body. The pulse signal is transmitted through the human body to realize the intra-body communication. The control module analyzes the pulse signal transmitted through the human body, so as to identify whether the tube body is inserted into the esophagus or trachea.

The above-mentioned embodiment is illustrative. It should be noted that for those skilled in the art, any variations and modifications without departing from the spirit of the disclosure should fall in the scope of the present disclosure.

What is claimed is:

1. An active inserted gastric tube with an intra-body communication function, comprising:
a tube body;
a pulse module;
an intra-body communication module; and
a control module;
wherein the pulse module is arranged at a front end of the tube, and is configured to generate a pulse signal; the intra-body communication module is configured to receive the pulse signal generated by the pulse module and transmit the pulse signal to the control module; and the control module is configured to analyze the received pulse signal;
the pulse module comprises a pulse generator, a first electrode and a first pulse amplifier; the pulse generator is electrically connected to the first pulse amplifier; the first pulse amplifier is electrically connected to the first electrode; and the first electrode is arranged at the front end of the tube;
a channel is arranged in a wall of the tube; a wire is arranged inside the channel; the first pulse amplifier is electrically connected to the first electrode through the wire; the first electrode is circular; and the first electrode is coated on an edge of the front end of the tube;
the intra-body communication module comprises a human body surface, an esophagus and a trachea; and the tube is insertable into the esophagus; and the control module is in communication with the human body surface; and
the control module comprises a microprocessor, a second pulse amplifier and a second electrode; the second electrode is a sheet, and is attached to the human body surface; the second electrode is electrically connected to the second pulse amplifier; the second pulse amplifier is electrically connected to the microprocessor; the microprocessor is configured to analyze a response curve of a resistance-capacitance (R-C) network activated by a step function; and the R-C network is an electrical impedance of the human body.
